(19) United States
(12) Patent Application Publication
(10) Pub. No.: US 2025/0046463 A1
(43) Pub. Date: Feb. 6, 2025

(54) SYSTEMS AND METHODS FOR GENERATING HEALTH REPORTS BASED ON VETERINARY ORAL CARE HEALTH TEST

(71) Applicant: Mars, Incorporated, McLean, VA (US)

(72) Inventors: Marie-Louise Amanda BENNETT, Melton Mowbray (GB); Gordon Craig CAMERON, Franklin, TN (US); Lucy Jane HOLCOMBE, Melton Mowbray (GB); Philip Martin MCGENITY, Melton Mowbray (GB); Avika Kishorlal RUPARELL, Melton Mowbray (GB); Corryn Victoria WALLIS, Melton Mowbray (GB); Amanda Elizabeth DAVIES, Melton Mowbray (GB)

(21) Appl. No.: 18/709,169

(22) PCT Filed: Nov. 11, 2022
(86) PCT No.: PCT/US2022/079689

§ 371 Date: May 10, 2024

Related U.S. Application Data
(60) Provisional application No. 63/278,689, filed on Nov. 12, 2021.

(51) Int. Cl. G16H 50/30; G16H 10/60

(52) U.S. Cl. CPC G16H 50/30; G16H 10/60

(57) ABSTRACT

Methods, systems, and non-transitory computer readable mediums are disclosed for generating health reports. Pet data associated with an entity is received from one or more data sources. Genetic test results data is received from a genetic test results database. The pet data received from the one or more data sources is analyzed with the genetic test results data received from the genetic test results database using an interpretation logic data structure. A result value associated with the pet data and the genetic test results data is determined based on the analysis. One or more result statements associated with the result value are determined and one or more health reports based on the one or more result statements are generated.

### CROSS-REFERENCE TO RELATED APPLICATION

This is a National Stage Application under 35 U.S.C. § 371 of International Application No. PCT/US2022/079689, filed on Nov. 11, 2022, which claims priority to U.S. Patent Application No. 63/278,689, filed on Nov. 12, 2021, the entireties of which are incorporated herein by reference.

### TECHNICAL FIELD

Various embodiments of the present disclosure relate generally to systems and methods for generating statements related to pet oral health, and more particularly, to systems, computer-implemented methods, and non-transitory computer readable mediums for analyzing pet data with genetic test results using interpretation logic or machine learning techniques to generate statements for pet oral health reports.

### BACKGROUND

Pet oral health and diseases resulting from poor oral hygiene (e.g., gum disease, periodontal disease) in pets are assessed and treated, if necessary, by routine visits to the veterinarian. Yet, symptoms of periodontal disease in pets often remain untreated due to infrequent visits to the veterinarian, improper oral care, and lack of knowledge regarding pet dental heath from pet owners. Failure to address the symptoms of periodontal disease increases the probability of disease progression.

Furthermore, despite its prevalence as the number one health problem in pets, periodontal disease remains largely underdiagnosed. Nevertheless, with early detection and proper oral care and/or treatment, periodontal disease in pets is preventable. Therefore, a need exists for a system and method for analyzing a comprehensive dataset from a pet to assess the pet's periodontal disease status and generating oral health reports for both pet owners and veterinarians based on the analysis.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, computer-implemented methods, systems, and non-transitory computer readable mediums are disclosed for analyzing pet data with genetic test results associated with a pet, determining a result value based on the analysis, and determining one or more result statements associated with the result value. The computer-implemented methods, systems, and non-transitory computer readable mediums of the present disclosure may also generate one or more health reports based on the one or more results statements.

In one aspect, an exemplary computer-implemented method may include receiving, by one or more first processors, pet data associated with an entity from one or more data sources and receiving, by the one or more first processors, genetic test results from a genetic test results database. The method may also include analyzing, by the one or more first processors, the pet data received from the one or more data sources with the genetic test results data received from the genetic test results database using an interpretation logic data structure. Subsequent steps may include: determining, by the one or more first processors, a result value associated with the pet data and the genetic test results data based on the analysis; determining, by the one or more first processors, one or more result statements associated with the result value; and generating, by the one or more first processors, one or more health reports based on the one or more result statements.

In some embodiments, the interpretation logic data structure may include a plurality of results values and a combination of result statements associated with each result value. The interpretation logic data structure may define a genetic test results range, a periodontal disease risk range, and a halitosis status for each of the plurality of result values. In at least one example, each of the combination of result statements may include at least one of a diagnosis or an oral care recommendation. In some examples, the one or more health reports include a pet owner report and/or a veterinarian report.

In another aspect, an exemplary system may include a genetic test results database and an interpretation logic data structure. The system may also include a periodontal disease risk determination component. The periodontal disease risk determination component may be executed by one or more first processors. Said periodontal disease risk determination component may be configured to receive one or more periodontal disease attributes from a database or one or more user devices and determine a periodontal disease risk level based on the analysis. The periodontal disease risk determination component may also be configured to generate periodontal disease risk data based on the determined periodontal disease risk level.

An exemplary system of the present disclosure may also include an analytics module executed by one or more second processors. The analytics module may be configured to receive pet data associated with an entity from one or more data sources, wherein the pet data includes at least the periodontal disease risk data from the periodontal disease risk component. The analytics module may be configured to receive genetic test results from the genetic test results database. The analytics module may also be configured to analyze the pet data received from the one or more data sources with the genetic test results received from the genetic test results database using the interpretation logic data structure and to determine a result value associated with the pet data and the genetic test results based on the analysis.

In yet another aspect, an exemplary non-transitory computer readable medium stores instructions that may be executed by one or more processors. When executed by one or more processors, the stored instructions of the exemplary non-transitory computer readable medium may cause the one or more processors to receive pet data associated with an entity from one or more data sources and to receive genetic test results data from a genetic test results database. The instructions may also cause the one or more processors to analyze the pet data received from the one or more data sources with the genetic test results data received from the genetic test results database using an interpretation logic data structure. The instructions may cause the one or more processors to determine a result value associated with the pet data and the genetic test results data based on the analysis and to determine one or more results associated with the result value. The stored instructions may also cause the one or more processor to generate one or more health reports based on the one or more result statements.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a block diagram of an exemplary system for analyzing data associated with a pet, according to one or more embodiments.

FIG. 2 depicts a flowchart illustrating an exemplary process for generating periodontal disease (PD) risk data, according to one or more embodiments.

FIG. 3 depicts an exemplary interpretation logic data structure for determining one or more result statements based on the data associated with a pet, according to one or more embodiments.

FIG. 4 depicts a flowchart illustrating exemplary steps performed by an analytics module using an interpretation logic data structure and pet data received from various sources, according to one or more embodiments.

FIG. 5 depicts a flowchart illustrating an exemplary method of training a machine learning model to determine a classification based on the data associated with a pet, according to one or more embodiments.

FIG. 6 depicts a flowchart illustrating an exemplary method of using a machine learning model to determine one or more result statements based on data associated with a pet, according to one or more embodiments.

FIG. 7 illustrates an implementation of a computer system that may execute techniques presented herein.

### DETAILED DESCRIPTION

Various embodiments of the present disclosure relate generally to statements related to pet oral health, such as statements based on analyzed pet metadata and genetic test results data. More particularly, various embodiments of the present disclosure relate to systems, computer-implemented methods, and non-transitory computer readable mediums for analyzing pet data with genetic test results using an interpretation logic data structure or machine learning techniques to generate statements for pet oral health reports. In a preferred embodiment, quantitative polymerase chain reaction (qPCR) results from a qPCR genetic test are analyzed with pet data using an interpretation logic data structure or machine learning techniques to generate statements for pet oral health reports.

As discussed above, despite being preventable, periodontal disease is prevalent in pets, often caused by poor oral hygiene as well as the failure to detect and/or treat early signs of the disease. In some instances, a veterinarian may fail to properly diagnose and treat periodontal disease in its early stages, while pet owners may fail to practice or maintain proper preventative measures. A variety of methods for assessing a pet's oral health exist, including but not limited to visual examination, oral testing, and predictive modeling. However, the previous methods utilize a singular approach, failing to evaluate all possible factors that contribute to periodontal disease and to advise accordingly.

Therefore, the embodiments of the present disclosure are directed to solving, mitigating, or rectifying the above-mentioned issues by analyzing a pet's oral health based on a combination of environmental/external factors (e.g., oral microbiome, oral care routine) and biological factors (e.g., age, weight, signs of periodontal disease) and then synthesizing the results from the analysis into statements based on the analyzed result for both pet owners and veterinarians. The systems and methods of the present disclosure may address the above-mentioned issues by receiving data related to a pet from a variety of sources, including genetic test results from genetic testing of an oral sample collected from the pet and periodontal disease risk data determined from user (e.g., pet owner and/or veterinarian) input data. Genetic test results of the present disclosure may be obtained from quantitative polymerase chain reaction (qPCR) testing, sequencing (e.g., high-throughput sequencing, nanopore sequencing, and single-molecule real-time sequencing), or other means for determining genetic sequences or genetic traits. Once the data, including additional data related to halitosis, is received, the data may be analyzed by an analytics module, using an interpretation logic data structure. The interpretation logic data structure may be used to associate the genetic test results, the periodontal disease risk data, and halitosis data with a result value. The result value from the interpretation logic data structure may then be used to determine one or more result statements based on the result value. The present disclosure allows statements providing a diagnosis and/or oral care recommendations to be included in pet owner and/or veterinarian reports, based on a result value determined by the analysis of genetic test results with various other types of biological and/or environmental data associated with a pet.

The present disclosure provides systems, methods, non-transitory computer readable mediums, and/or devices configured to receive and analyze a pet's biological and environmental data (e.g., periodontal disease risk data and halitosis data) with the pet's genetic test results (e.g., quantitative polymerase chain reaction (qPCR) results), determine a result value based on the analysis, determine one or more result statements based on the determined result value, and generate one or more health reports based on the one or more result statements. The following embodiments describe systems, computer-implemented methods, and non-transitory computer readable mediums for generating one or more statements related to pet oral health based on a result value determined from analyzed pet data.

Specifically, data associated with a pet may be analyzed with test results from the pet using an interpretation logic data structure. The pet data may include periodontal disease risk data, previously determined from periodontal disease risk attributes of the pet, and halitosis data. The test results may include genetic test results obtained from genetic testing. In a preferred embodiment, the genetic test results may include qPCR results obtained from a quantitative polymerase chain reaction test performed on an oral sample collected from the pet. The interpretation logic data structure may contain genetic test (e.g., qPCR) result ranges, periodontal disease risk ranges, and halitosis statuses as well as a result value associated with various combinations of a genetic test (e.g., qPCR) result range, periodontal disease risk range, and a halitosis status. The interpretation logic data structure may also include result statements associated with each result value.

The interpretation logic data structure is used to compare the pet data (e.g., biological data of a pet), including periodontal disease risk data and halitosis data, and the genetic test results of the pet with the respective interpretation logic data structure categories (e.g., genetic test results range, periodontal disease risk ranges, and halitosis status) and to determine an associated result value. The result value may be used to determine one or more statements associated with the result value. Each statement may be classified as a statement for either a pet owner health report or a veterinarian health report. The statements may include at least of a diagnosis or an oral care recommendation for pet owner and/or veterinarian health reports.

In at least one embodiment of the present disclosure, a machine-learning model may determine one or more result statements related to pet oral health. A machine-learning model may be trained to generate a classification based on sample pet data and sample genetic test results from pets. The trained machine-learning model may then be used to analyze pet data and genetic test results from a specific pet, determine a classification for the pet based on the prepared pet data and genetic test results, and determine one or more result statements based on the classification. The one or more result statements may include statements for pet owner and/or veterinarian health reports as discussed above.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” “includes,” “including,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The terms “pet” and “household pet” as used in accordance with the present disclosure can refer to, without limitation, domesticated or tamed animals such as, e.g., dogs, cats, rabbits, horses, and the like.

The term “pet owner” may include, for example, without limitation, any person, organization, and/or collection of persons that owns and/or provides food and shelter for a pet. For example, a “pet owner” may include a pet adopter, a pet caretaker, a pet caregiver, and an animal shelter.

The term “veterinarian” may include, for example, without limitation, any person, organization, and/or collection of persons that provides medical care to a pet.

For example, a “veterinarian” may include a veterinary technician, a veterinary personnel, and a veterinarian practitioner.

The terms “canine” and “dog” may include, for example, without limitation, recognized dog breeds (some of which may be further subdivided). For example, the recognized dog breeds may include afghan hound, Airedale, Akita, Alaskan malamute, basset hound, beagle, Belgian shepherd, bloodhound, border collie, border terrier, borzoi, boxer, bulldog, bull terrier, cairn terrier, Chihuahua, chow, cocker spaniel, collie, corgi, dachshund, Dalmatian, Doberman, English setter, fox terrier, German shepherd, golden retriever, great dane, greyhound, griffon bruxellois, Irish setter, Irish wolfhound, King Charles spaniel, Labrador retriever, lhasa apso, mastiff, newfoundland, old English sheepdog, Papillion, Pekingese, pointer, Pomeranian, poodle, pug, Rottweiler, St. Bernard, saluki, Samoyed, schnauzer, Scottish terrier, Shetland sheepdog, shih tzu, Siberian husky, Skye terrier, springer spaniel, West Highland terrier, whip companion, Yorkshire terrier, etc.

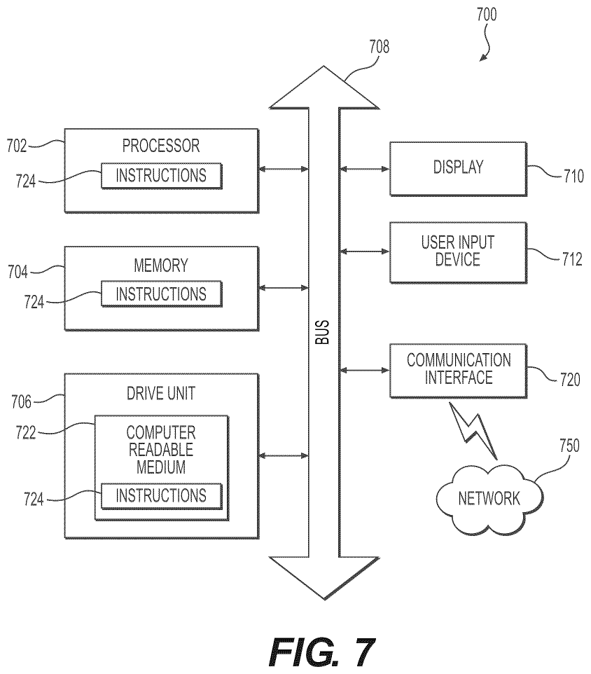

The terms “device” and “user device” may include, for example, without limitation, any electronic equipment, controlled by a central processing unit (CPU), for inputting information or data and displaying a user interface. A user device can send or receive signals, such as via a wired or wireless network, or can process or store signals, such as in memory as physical memory states. A device or user device as used in the present disclosure may include: a desktop computer; a mobile computer (e.g., a tablet computer, a laptop computer, or a notebook computer); a smartphone; a wearable computing device (e.g., smart watch); or the like, consistent with the computing device shown in FIG. 7.

As used herein, the terms “pet data” or “pet metadata” may include, for example, without limitation, biological data, such as, any one or combination of certain biological information or attributes of a pet including at least its breed, age, size, weight, body condition, shape of head (e.g., skull shape), predicted size category, predicted weight as an adult, and/or oral health data related to common symptoms of gum or periodontal disease. The oral health data related to common symptoms of gum or periodontal disease may include, the presence of bleeding gums, inflamed (i.e. reddened) gums, swollen (e.g., puffy) gums, tartar, plaque, clinical attachment loss (e.g., periodontal pocket, gingival recession, furcation exposure), tooth loss or missing teeth, alveolar bone loss, sensitive stomach, and/or halitosis, and/or medical history of gingivitis or periodontal disease. Other oral health data may include the bacterial composition of plaque. Pet data may also include environmental data (e.g., external factors). Environmental data may include information regarding a pet's oral care routine, such as the frequency of tooth brushing and/or the use of dental treats, oral rinses, oral gels, and chew toys. Other environmental data may include changes in pet behavior (e.g., eating habits and pawing at the face), which may indicate gum or periodontal disease. Additionally, for example, the pet data may include answers to questions pertaining to, but not limited to, the biological information, environmental/external factors, and attributes discussed above. For the purposes of the current disclosure, certain other types of biological information, such as genetic test results, will not be referred to as pet data or pet metadata.

As used herein, the term “periodontal disease attribute” may include, for example, an attribute or characteristic of a pet that may contribute more or less to a pet's predisposition to periodontal disease. Examples of periodontal disease attributes include, but are not limited to, breed, age, size, weight, body condition, shape of head (e.g., skull shape), predicted size category, predicted weight as an adult, amount of plaque/tartar, and the bacterial composition of plaque.

The term “oral sample” may include, for example, without limitation, plaque, saliva, or a sample of an oral fluid collected from the oral cavity of a pet.

The terms “genetic test” and “genetic testing” may include, for example, without limitation, quantitative polymerase chain reaction (qPCR), loop-mediated isothermal amplification, high throughput sequencing, nanopore DNA sequencing, single-molecule real-time sequencing, next generation sequencing, Illumina sequencing, or other means for determining genetic sequences and/or genetic traits.

As used herein, an “interpretation logic data structure” generally encompasses a data structure, such as a table, containing data associated with a plurality of outcome values. An “interpretation logic data structure” may also be referred to herein as an “interpretation logic data table.” The interpretation logic data table may include a table showing each possible input combination with a resultant output depending upon the combination of the inputs. The output may include outcome or result values and outcome or result statements associated with the values.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. Any suitable type of training may be used, e.g., stochastic, gradient boosted, random seeded, recursive, epoch or batch-based, etc.

The term “diagnosis” may include, for example, without limitation, the recognition and/or identification of a disease, the prediction of the course of a disease, as well as a conclusion with respect to a risk level associated with a disease (e.g., low risk, medium risk, high risk).

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

In certain non-limiting embodiments, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors, such as an elastic computer cluster, and associated network and storage devices, as well as operating software and one or more database systems and application software that support the services provided by the server. The server, for example, can be a cloud-based server, a cloud-computing platform, or a virtual machine. Servers can vary widely in configuration or capabilities, but generally a server can include one or more central processing units and memory. A server can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For some non-limiting embodiments, a “network” should be understood to refer to a network that may couple devices so that communications can be exchanged, such as between a server and a user device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

Furthermore, devices or user devices, such as computing devices or other related electronic devices can be remotely coupled to a network, such as via a wired or wireless line or link, for example.

In certain non-limiting embodiments, a “wireless network” should be understood to couple user devices with a network. A wireless network can include virtually any type of wireless communication mechanism by which signals can be communicated between devices, between or within a network, or the like. A wireless network can employ standalone ad-hoc networks, mesh networks, wireless land area network (WLAN), cellular networks, or the like. A wireless network may be configured to include a system of terminals, gateways, routers, or the like coupled by wireless radio links, or the like, which can move freely, randomly, or organize themselves arbitrarily, such that network topology can change, at times even rapidly.

A wireless network can further employ a plurality of network access technologies, including Wi-Fi, Long Term Evolution (LTE), WLAN, Wireless Router (WR) mesh, or 2nd, 3rd, 4th, 5th generation (2G, 3G, 4G, or 5G) cellular technology, or the like. Network access technologies can allow wide area coverage for devices, such as user devices with varying degrees of mobility, for example.

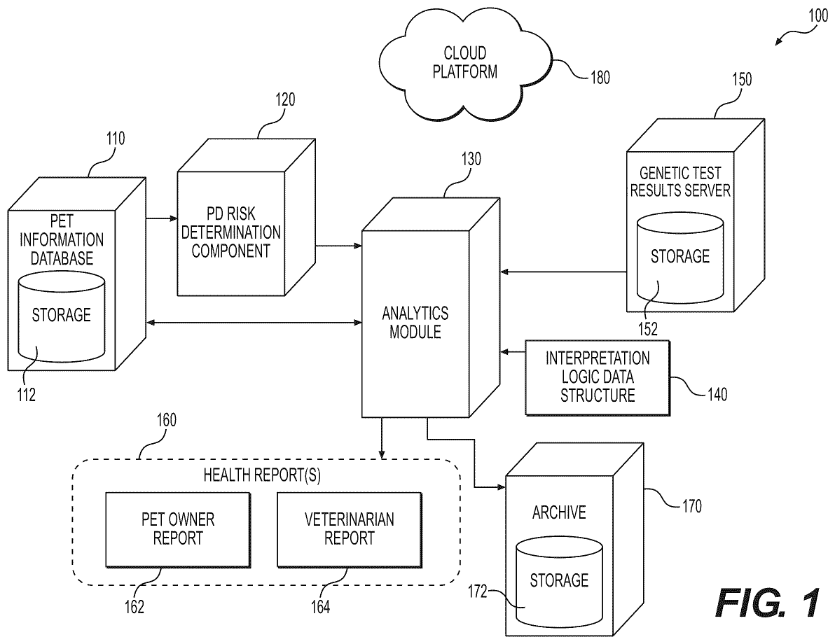

Referring now to the appended drawings, FIG. 1 shows an exemplary embodiment of a system 100 for analyzing data associated with a pet. In general, system 100 may comprise pet information database 110, PD risk determination component 120, analytics module 130, interpretation logic data structure 140, genetic test results server 150, health report(s) 160, archive 170, and cloud platform 180.

System 100 may be implemented on a cloud platform 180, allowing for the transmission or sharing of data between each of pet information database 110, PD risk determination component 120, analytics module 130, interpretation logic data structure 140, genetic test results server 150, health report(s) 160, and archive 170 in a cloud environment.

Pet information database 110 comprises storage 112. Storage 112 in pet information database 110 may contain pet data (e.g., biological and environmental data) associated with a pet. One or more users may access pet information database 110 by a server via one or more user devices. The one or more users may include a pet owner and a veterinarian. The one or more users may use one or more user devices to input pet data associated with a pet. A user device may be a device consistent with the computing device depicted in FIG. 7, such as a desktop computer, a tablet, a laptop, a smart phone, a smart watch, etc.

The pet data associated with a pet and input by one or more users into pet information database 110 may include one or more of the following attributes of a pet: breed, age, size, weight, body condition, shape of head (e.g., skull shape), predicted size category, and predicted weight as an adult, amount of plaque/tartar, and the bacterial composition of plaque. Additional pet data which may be input by the one or more users and stored in pet information database 110 may include oral health data pertaining to common symptoms of periodontal disease and information pertaining to a pet's oral care routine. For example, the one or more users may input information regarding whether the pet has swollen, inflamed, reddened, and/or bleeding gums, a change in behavior (e.g., change in eating habits or pawing at the face), a sensitive stomach, halitosis, tooth loss, clinical attachment loss, alveolar bone loss, plaque, or tartar build-up, as well as the frequency of tooth brushing and use of dental treats, oral rinses, and/or oral gels. The pet data associated with a pet may be stored in storage 112 of pet information database 110.

In some embodiments, both a pet owner and a veterinarian may input pet data associated with a pet in pet information database 110. In other embodiments, only one of a pet owner and a veterinarian may input pet data associated with a pet in pet information database 110. In at least some embodiments, the one or more users may be prompted to input at least two of the following attributes of a pet in pet information database 110: breed, age, size, weight, body condition, shape of head (e.g., skull shape), predicted size category, predicted weight as an adult, amount of plaque/tartar, and the bacterial composition of plaque. In some examples, the one or more users may be prompted to input a halitosis status of absent or present in pet information database 110.

Pet information database 110 may transmit pet data associated with a pet to PD risk determination component 120. Pet information database 110 may also transmit pet data associated with a pet to analytics module 130. In some embodiments, pet information database 110 may also receive data, such as one or more results statements related to a pet's oral health, from analytics module 130.

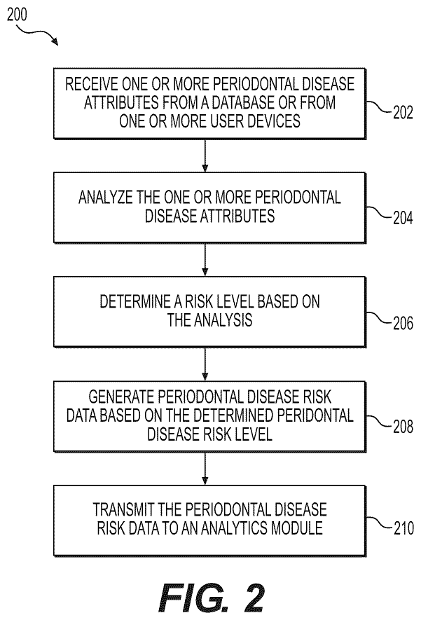

PD risk determination component 120 may receive pet data associated with a pet from pet information database 110 as discussed above. PD risk determination component 120 may be deployed in an application programming interface (API). PD risk determination component 120 may also transmit periodontal disease risk data to analytics module 130. A more detailed description of PD risk determination component 120 is provided further below in reference to FIG. 2. PD risk determination component 120 as described herein may also be used on other applications (e.g., websites) outside of system 100.

Genetic test results server 150 comprises storage 152. A user may input genetic test results, which are obtained from a genetic test performed on an oral sample collected from a pet (e.g., quantitative polymerase chain reaction (qPCR) testing), onto genetic test results server 150 via user device. For example, the user may input the genetic test result for a pet as a numerical value between 0 and 1. Alternatively, the user may input the genetic test result for a pet based on binary data (i.e., the presence or absence of a microorganism (e.g., bacteria)). The genetic test results may be stored in storage 152. Genetic test results server 150 may transmit genetic test results to analytics module 130.

Analytics module 130 may receive pet data associated with a pet from one or more data sources in system 100 and genetic test results data associated with the pet from genetic test results server 150. For example, analytics module 130 may receive pet data associated with a pet directly from pet information database 110 as well as from PD risk determination component 120. In some embodiments, select pet data associated with a pet may be transmitted from pet information database 110 to PD risk determination component 120 for analysis and the periodontal disease risk data that is generated based on the analysis may be transmitted to analytics module 130. In some embodiments, certain types of pet data, such as, e.g., halitosis data, may be transmitted directly from pet information database 110 to analytics module 130, without first being transmitted to PD risk determination component 120.

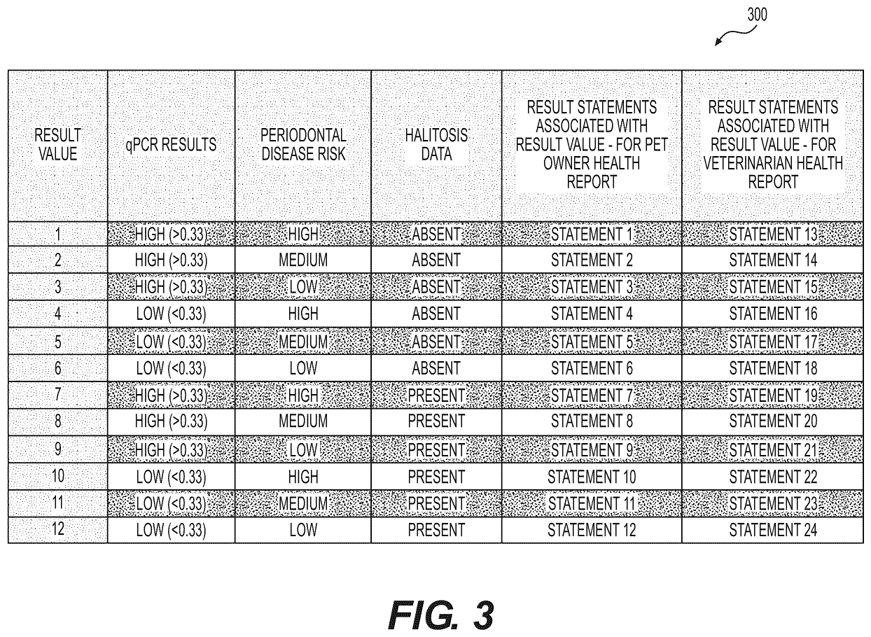

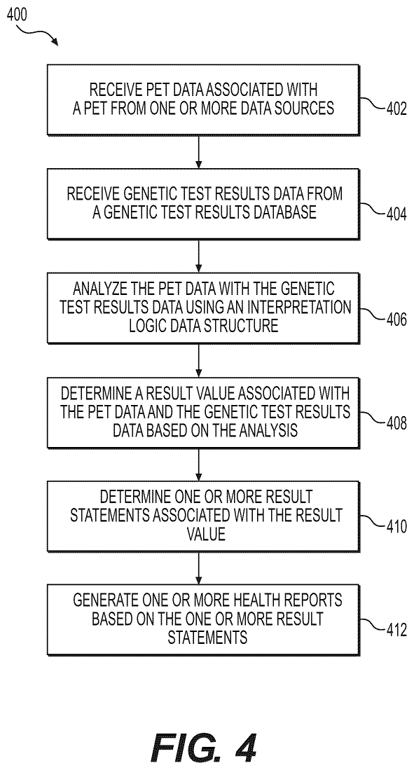

Analytics module 130 may use interpretation logic data structure 140 to analyze the pet data received with the genetic test results received. A more detailed description of interpretation logic data structure 140 is provided further below in reference to FIG. 3. Analytics module 130 may determine one or more statements related to a pet's oral health based on the analysis of the pet's pet data with the pet's genetic test results using interpretation logic data structure 140 and may generate health report(s) 160 containing the one or more statements. Health report(s) 160 may include pet owner report 162 and/or veterinarian report 164. Analytics module 130 may also transmit/store the one or more statements generated to archive 170. Archive 170 may store the analyzed data received from analytics module 130 in storage 172. Further, a more detailed description of analytics module 130 is provided further below in reference to FIG. 4.

It should be noted that, although system 100 depicts genetic test results server 150 for storing genetic test results, the system may comprise a server and storage for results obtained from any laboratory test conducted on an oral sample collected from a pet. In some embodiments, a server and storage may contain results from testing conducted on a biological sample collected from a pet, other than an oral sample (e.g., saliva or plaque), such as blood, stool, urine, hair, and tissue. In a preferred embodiment, the system may comprise a server and storage results obtained from quantitative polymerase chain reaction (qPCR) testing of an oral sample (e.g., saliva or plaque) collected from a pet.

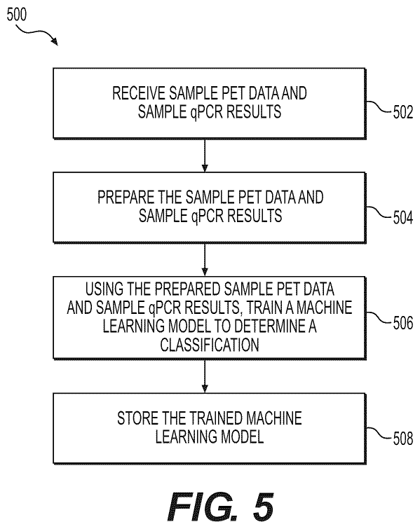

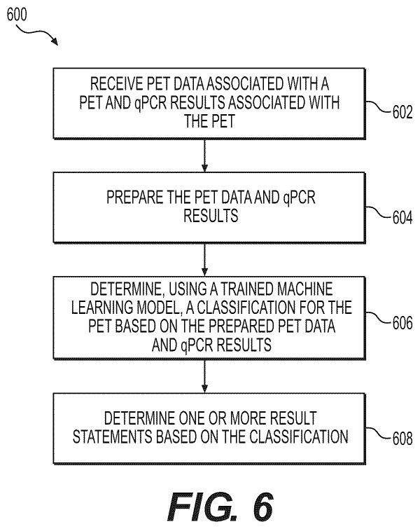

It should also be noted that, although system 100 depicts the use of interpretation logic data structure 140 by analytics module 130, in some embodiments, analytics module 130 may use a machine-learning model. In these embodiments, a trained machine learning model may be stored and used by analytics module 130. A more detailed description of machine-learning is provided further below in reference to FIGS. 5 and 6.

FIG. 2 depicts a flowchart illustrating exemplary process 200 performed by PD risk determination component 120 for generating periodontal disease (PD) risk data. In step 202, PD risk determination component 120 may receive one or more periodontal disease attributes from a database or from one or more user devices. For example, PD risk determination component 120 may receive one or more periodontal disease attributes of a pet from pet information database 110. Exemplary periodontal disease attributes of a pet that may be received and analyzed by PD risk determination component 120 include: breed, age, size, weight, body condition, shape of head (e.g., skull shape), predicted size category, predicted weight as an adult, amount of plaque/tartar, the bacterial composition of plaque, and oral health data related to common symptoms of gum or periodontal disease, such as the presence of bleeding gums, swollen gums, change in eating habits, sensitive stomach, medical history of gingivitis or periodontal disease, and the like. Some of the periodontal disease attributes, such as age and weight, may be provided by a pet owner, while other periodontal disease attributes, such as predicted size category, amount of plaque/tartar, and the bacterial composition of plaque, may be provided by a veterinarian. In some embodiments, PD risk determination component 120 may receive the one or more periodontal disease attributes from one or more user devices. For example, PD risk determination component 120 may prompt one or more users (e.g., a pet owner, a veterinarian) to input one or more periodontal disease attributes via a user interface rendered on one or more user devices.

In step 204, PD risk determination component 120 may analyze the one or more periodontal disease attributes received in step 202. In step 206, PD risk determination component 120 may determine a risk level based on the analysis of step 204. In some embodiments, PD risk determination component 120 may operate using a model built based on a plurality of pet attributes that are associated with a risk level for periodontal disease. Some pet attributes that may be used in the model for PD risk determination component 120 include, but are not limited to, amount of plaque and/or tartar, weight, breed, size, and age group. For example, certain breeds of dogs may be more susceptible to periodontal disease than others and thus, have a higher risk of periodontal disease.

In an example, where PD risk determination component 120 receives data related to the breed of a pet, the breed is analyzed in step 204 and the pet is assigned a probability for periodontal disease based on the breed attribute. However, as explained above, it should be understood that the PD risk determination component 120 may consider a combination of attributes to determine a probability for periodontal disease. The probability may be a number between 0 and 1. In step 206, PD risk determination component 120 may associate the analyzed probability of periodontal disease from step 204 with a risk level. Exemplary risk levels include high, medium, and low. For example, a low risk level may be associated with a probability of 0.33 or less. A medium risk level may be associated with a probability of greater than 0.33 and less than 0.67. A high risk level may be associated with a probability of 0.67 or greater.

In step 208, PD risk determination component 120 may generate periodontal disease risk data based on the periodontal disease risk level determined in step 206. For example, if the one or more pet attributes received for a pet are associated with a medium risk level, then the periodontal disease risk data generated in step 208 will be based on a medium risk level and classified as “medium.” If the one or more pet attributes received for a pet are associated with a low risk level, then the periodontal disease risk data generated in step 208 will be based on a low risk level and classified as “low.” If the one or more pet attributes received for a pet are associated with a high risk level, then the periodontal disease risk data generated in step 208 will be based on a high risk level and classified as “high.” In step 210, PD risk determination component 120 may transmit the periodontal disease risk data for the pet to analytics module 130.

FIG. 3 illustrates an exemplary interpretation logic data table 300, which may be used for interpretation logic data structure 140. Exemplary interpretation logic data table 300 is based on quantitative polymerase chain reaction (qPCR) test results and embodiments where qPCR testing is used as the genetic test performed on the oral sample collected from a pet. For instance, table 300 contains columns for a plurality of result values, qPCR results, periodontal disease risk categories, halitosis data, result statements associated with a result value for a pet owner health report, and result statements associated with a result value for a veterinarian health report. The “Result Value” column contains a plurality of result values ranging from 1 to 12. Each result value is associated with a qPCR result, a periodontal disease risk, a halitosis status, a result statement for a pet owner health report, and a result statement for a veterinarian health report. Table 300 shows that a result value of 1 corresponds to a high qPCR result wherein the value is greater than 0.33, high periodontal disease risk, an absent halitosis status (i.e., no halitosis), statement 1 for a pet owner health report, and statement 13 for a veterinarian health report. The interpretation logic data structure 140, e.g., interpretation logic data table 300, may be added, updated, and/or removed by a user (e.g., an administrator) using a user device. In one embodiment, a computing device storing the interpretation logic data structure 140 may be configured to provide a user interface on a user device, allowing for creation, modification, or deletion of one or more interpretation logic data structures 140 by a user using the user device.

In examples where a genetic test other than qPCR testing is conducted on an oral sample collected from a pet, the interpretation logic data structure 140 may be based on the test results obtained from the specific genetic test performed. For example, when a genetic test, such as high throughput sequencing or nanopore sequencing is used, an interpretation logic data table similar to interpretation logic data 300 may be created based on the high throughput sequencing or nanopore sequencing results.

Analytics module 130 may use interpretation logic data structure 140, which may be based on table 300, to analyze the pet data associated with a pet, including periodontal disease risk data and halitosis data with the qPCR results associated with the pet. Table 300 may be used to compare the qPCR results, the periodontal disease risk data, and the halitosis data associated with a pet and received by analytics module 130 with the ranges and/or statuses of the respective categories in the table, in order to determine a corresponding result value. As discussed above, according to 300, data received from a pet corresponding to a qPCR result of greater than 0.33 (High), a high periodontal disease risk, and an absent halitosis status, would achieve a result value of 1. Table 300 associates a result value of 1 with (result) statement 1 and (result) statement 13. Statement 1 and/or Statement 13 correspond to statements for a pet owner health report and a veterinarian health report, respectively.

FIG. 4 depicts process 400 which may be performed by analytics module 130, using interpretation logic data structure 140 and pet data received from various sources. In step 402, analytics module 130 receives pet data associated with a pet from one or more data sources. In some embodiments, analytics module 130 receives pet data associated with a pet from pet information database 110 as well as periodontal disease risk data associated with the pet from PD risk determination component 120. In one embodiment, the pet data received directly from pet information database 110 may include halitosis data. As discussed above, the periodontal disease risk data associated with a pet and generated by PD risk determination component 120 may be based on certain pet data associated with the pet (e.g., one or more periodontal disease attributes) received at PD risk determination component 120 from pet information database 110. The pet data associated with a pet and received by analytics module 130 may include periodontal disease risk data associated with a rating or level of high, medium, or low, as well as halitosis data associated with a status of absent or present.

In step 404, analytics module 130 receives genetic test results data associated with the pet from a genetic test results database. For example, analytics module 130 may receive genetic test results data associated with the pet from storage 152 of genetic test results server 150. The genetic test results data associated with the pet may be received in the form of a numerical value between 0 and 1.

In step 406, the analytics module 130 analyzes the pet data and genetic test results data associated with a pet using interpretation logic data structure 140. In some embodiments, interpretation logic data table 300 is used for interpretation logic data structure 140. The analytics module 130 may compare the genetic test results data, the periodontal disease risk data, and the halitosis data received for the pet with the ranges and/or statuses of the respective categories in interpretation logic data table 300. In step 408, analytics module 130 determines a result value based on the analysis from step 406.

After a result value has been determined, in step 410 the analytics module 130 uses interpretation logic data structure (e.g., interpretation logic data table 300) to determine one or more results statements associated with the result value. In step 412, analytics module 130 generates one or more health reports 160, based on the one or more result statements.

In an exemplary embodiment of the present disclosure, a pet owner or a pet's veterinarian may collect an oral sample from a pet for quantitative polymerase chain reaction (qPCR) testing. A qPCR assay may be conducted on the oral sample in a laboratory and the qPCR results obtained for the pet may be given a numerical value between 0 and 1, based on a relative abundance of biomarker species to total bacterial load. For example, the qPCR result for the pet may be 0.40. Alternatively, a different numerical scale outside of the range of 0 and 1 may be provided for a raw qPCR value where the raw qPCR value is not limited to between 0 and 1. A user, such as a lab employee, may access genetic test (qPCR) results server 150 via a user device and may input the genetic test (qPCR) results data associated with the pet via the user device. Genetic test (qPCR) results server 150 may transmit the genetic test (qPCR) results data associated with the pet to analytics module 130. Either before or after the oral sample is collected from the pet and tested, the pet owner and/or the veterinarian may access pet information database 110 via their respective user devices and may input pet data associated with the pet. For example, the pet owner may input the pet's age and that no dental treats are used in the pet's oral care routine. The veterinarian may input the pet's weight and a halitosis status of present. Pet information database 110 may store the pet data associated with the pet.

In one embodiment, pet information database 110 may transmit certain pet data such as, e.g., the halitosis status of the pet, directly to analytics module 130. PD risk determination component 120 may receive other types of pet data associated with the pet (periodontal disease attributes) such as, e.g., age and weight, from pet information database 110. PD risk determination component 120 may analyze the periodontal disease attributes (e.g., age and weight) of the pet and may assign a probability for obtaining periodontal disease, between 0 and 1, based on the analysis. The probability may be associated with a risk category of high, medium, or low. In cases where PD risk determination component 120 determines a medium risk level based on the probability being greater than 0.33 and less than 0.67, PD risk determination component 120 may generate periodontal disease risk data associated with the pet based on the medium risk level.

In one embodiment, analytics module 130 may receive the periodontal disease risk data associated with the pet, the halitosis data associated with the pet, and the genetic test (e.g., qPCR) results data associated with the pet. Analytics module 130 may use interpretation logic data structure 140 to analyze the received data. In an example, the interpretation logic data structure 140 may be used to determine that a qPCR result of 0.40, a periodontal disease risk of medium, and a halitosis status of present, correlates to a result value of 8. Interpretation logic data structure 140 associates result statement 8 from the pet owner health report category and result statement 20 from the veterinarian health report category with result value 8. According to this example, when a result value of 8 is determined, analytics module 130 may generate pet owner report 162 based on result statement 8 and/or a veterinarian report 164 based on result statement 20.

Each result statement may contain at least one of a diagnosis and an oral care recommendation. As such, pet owner report 162 based on a result statement and/or veterinarian report 164 based on a result statement may contain at least one of a diagnosis and an oral care recommendation. In the exemplary embodiment, pet owner report 162 may contain at least one of a diagnosis and an oral care recommendation associated with result statement 8 and veterinarian report 164 may contain at least one of a diagnosis and an oral care recommendation associated with result statement 20.

An exemplary health report containing at least one diagnosis may explain that a pet is at either high, medium, or low risk for having gum disease. When the pet is at high or medium risk for having gum or periodontal disease, the health report may also provide additional information predicting the progression of the disease. In some embodiments where a veterinarian report is generated, the veterinarian report may only contain information pertaining to the diagnosis. In other embodiments, the veterinarian report may also contain a recommended treatment plan for the veterinarian, in addition to a diagnosis.

An exemplary health report containing at least one oral care recommendation may provide a recommended oral care routine or plan. For example, the report may recommend increased tooth brushing and use of dental treats/chews, oral rinses/gels, and water additives. The report may provide a recommended diet for the pet and a recommended date for scheduling the next visit to the veterinarian. The report may also provide information and statistics regarding the benefits of pet's having a healthy dental diet. In cases where the pet is diagnosed as having a high risk for periodontal disease, a report may recommend that the pet visit the veterinarian immediately.

FIG. 5 depicts a flowchart illustrating an exemplary method of training a machine learning model to determine a periodontal disease risk classification associated with a pet, according to one aspect of the present disclosure. The exemplary method 500 is based on quantitative polymerase chain reaction (qPCR) test results and embodiments where qPCR testing is used as the genetic test performed on the oral sample collected from a pet. Method 500 may be performed, by analytics module 130 or another suitable component in system 100, to train a machine learning model to determine a classification based on the data associated with a pet. For instance, method 500 may train a machine learning model using pet data and qPCR results. A machine learning model according to the present disclosure may be trained to make predictions pertaining to periodontal disease risk classification determination. In step 502, a machine learning model may receive sample pet data and sample qPCR results associated with pets. The sample pet data and sample qPCR results may come from a variety of sources as explained above. Each set of sample pet data and sample qPCR result(s) associated with a pet may be “labeled” with an appropriate classification (e.g., a result value contained in the interpretation logic data structure 140). In step 504, the sample pet data and the sample qPCR results may be “prepared” for model training. In step 506, the prepared sample pet data and sample qPCR results from pets may be used to train a machine learning model to determine a classification. In other words, the prepared sample pet data and sample qPCR results may be “fed” into a machine learning model, for the model to tune various parameters in order to arrive at the corresponding classifications. The machine learning model may also be tuned using feedback from human reviewers. In step 508, the trained machine learning model may be stored. For example, the trained machine learning model may be stored in the analytics module 130, storage 172 of archive 170 in system 100, and/or any suitable storage connected via network in system 100.

FIG. 6 depicts a flowchart illustrating an exemplary method of using a machine learning model to determine one or more result statements based on data associated with a pet, according to one or more embodiments. Method 600 may be performed in system 100, in place of or in addition to using interpretation logic data structure 140. As discussed above, analytics module 130 or any suitable component in system 100 may be configured to use a machine learning model. The exemplary method 600 is based on quantitative polymerase chain reaction (qPCR) test results and embodiments where qPCR testing is used as the genetic test performed on the oral sample collected from a pet.

In step 602, pet data associated with a pet and qPCR results associated with the pet may be received. In embodiments where analytics module 130 uses a machine learning model, the machine learning model may receive the pet data associated with a pet and the qPCR results associated with the pet by way of analytics module 130. For instance, pet data associated with a pet may be received from pet information database 110 and PD risk determination component 120. qPCR results associated with the pet may be received from genetic test results server 150.

In step 604, the machine learning model prepares the pet data and qPCR results, similar to the preparation process discussed above in reference to FIG. 5. The prepared data may be in format consumable by the machine learning model. In step 606, the trained machine learning model may determine a classification for the pet based on the prepared or analyzed pet data associated with the pet and qPCR results associated with the pet. The trained machine learning model, previously trained from sample pet data and sample qPCR results, may be configured to accurately determine a classification (e.g., a result value) for a pet.

The classification determined will be based on both the pet data (e.g., periodontal disease risk data and halitosis status) and qPCR results received for the pet. In step 608, the machine learning model may determine one or more result statements based on the classification determined in step 606. The one or more result statements determined in step 608, may be the same result statements from interpretation logic data structure 140 as shown in FIG. 3. For example, when the machine learning model in method 600 receives qPCR results for a pet in the range of less than 0.33 and biological data for the pet consistent with a low periodontal disease risk and the absence of halitosis, after determining a classification (e.g., a result of value of 6), the machine learning model may determine result statement 6 and result statement 18 based on the classification. Result statement 6 is used to generate a pet health report for a pet owner and result statement 18 is used to generate a pet health report for a veterinarian.

Once the one or more result statements are determined in step 608, analytics module 130, which may be configured to have a machine learning model component, may generate pet owner report 162 and veterinarian report 164 as discussed above.

Machine learning model processes 500 and 600 may also be used with genetic test results other than qPCR results. For instance, method 500 may train a machine learning model using pet data and genetic test results obtained from other methods of genetic testing (e.g., high throughput sequencing) and method 600 may use the trained machine learning model to determine a classification for a pet based on prepared pet data and genetic test results obtained from other methods of genetic testing (e.g., high throughput sequencing).

Machine learning model processes similar to those depicted in FIG. 5 and FIG. 6 may also be incorporated into other components of system 100, in addition to analytics module 130. In some examples of the present disclosure, PD risk determination component 120 may also use a machine learning model to determine the periodontal disease risk level. For example, a machine learning model used by PD risk determination component 120 may be trained by a process that is similar to method 500. The process of training a machine learning model used by the PD risk determination component may include receiving sample periodontal disease attributes data for pets, preparing the sample periodontal disease attributes data, using the prepared sample periodontal disease attributes data to train a machine learning model to determine a classification, and storing the trained machine learning model. The sample periodontal disease data for pets may be obtained from a variety of sources.

A machine learning model used by PD risk determination component 120 may operate according to a process that is similar to method 600. The machine learning model may receive one or more periodontal disease attributes data associated with a pet from one or more data sources, upon the data being prepared. For example, the machine learning model may receive one or more periodontal disease attributes data associated with a pet from pet information database 110. The one or more periodontal disease attributes data associated with the pet may be analyzed by the trained machine learning model to determine a classification for the pet. Lastly, the machine learning model, or PD risk determination component 120 utilizing the machine learning model, may determine a periodontal disease (PD) risk level based on the classification.

FIG. 7 illustrates an implementation of a computer system that may execute techniques presented herein. The computer system 700 can include a set of instructions that can be executed to cause the computer system 700 to perform any one or more of the methods or computer based functions disclosed herein. The computer system 700 may operate as a standalone device or may be connected, e.g., using a network, to other computer systems or peripheral devices.

In a networked deployment, the computer system 700 may operate in the capacity of a server or as a client user computer in a server-client user network environment, or as a peer computer system in a peer-to-peer (or distributed) network environment. The computer system 700 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 700 can be implemented using electronic devices that provide voice, video, or data communication. Further, while a single computer system 700 is illustrated, the term “system” shall also be taken to include any collection of systems or sub-systems that individually or jointly execute a set, or multiple sets, of instructions to perform one or more computer functions.

As illustrated in FIG. 7, the computer system 700 may include a processor 702, e.g., a central processing unit (CPU), a graphics processing unit (GPU), or both. The processor 702 may be a component in a variety of systems. For example, the processor 702 may be part of a standard personal computer or a workstation. The processor 702 may be one or more general processors, digital signal processors, application specific integrated circuits, field programmable gate arrays, servers, networks, digital circuits, analog circuits, combinations thereof, or other now known or later developed devices for analyzing and processing data. The processor 702 may implement a software program, such as code generated manually (i.e., programmed).

The computer system 700 may include a memory 704 that can communicate via a bus 708. The memory 704 may be a main memory, a static memory, or a dynamic memory. The memory 704 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 704 includes a cache or random-access memory for the processor 702. In alternative implementations, the memory 704 is separate from the processor 702, such as a cache memory of a processor, the system memory, or other memory. The memory 704 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 704 is operable to store instructions executable by the processor 702. The functions, acts or tasks illustrated in the figures or described herein may be performed by the programmed processor 702 executing the instructions stored in the memory 704. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the computer system 700 may further include a display unit 710, such as a liquid crystal display (LCD), an organic light emitting diode (OLED), a flat panel display, a solid-state display, a cathode ray tube (CRT), a projector, a printer or other now known or later developed display device for outputting determined information. The display 710 may act as an interface for the user to see the functioning of the processor 702, or specifically as an interface with the software stored in the memory 704 or in the drive unit 706.

Additionally or alternatively, the computer system 700 may include an input device 712 configured to allow a user to interact with any of the components of system 700. The input device 712 may be a number pad, a keyboard, or a cursor control device, such as a mouse, or a joystick, touch screen display, remote control, or any other device operative to interact with the computer system 700.

The computer system 700 may also or alternatively include a disk or optical drive unit 706. The disk drive unit 706 may include a computer-readable medium 722 in which one or more sets of instructions 724, e.g. software, can be embedded. Further, the instructions 724 may embody one or more of the methods or logic as described herein. The instructions 724 may reside completely or partially within the memory 704 and/or within the processor 702 during execution by the computer system 700. The memory 704 and the processor 702 also may include computer-readable media as discussed above.

In some systems, a computer-readable medium 722 includes instructions 724 or receives and executes instructions 724 responsive to a propagated signal so that a device connected to a network 750 can communicate voice, video, audio, images, or any other data over the network 750. Further, the instructions 724 may be transmitted or received over the network 750 via a communication port or interface 720, and/or using a bus 708. The communication port or interface 720 may be a part of the processor 702 or may be a separate component. The communication port 720 may be created in software or may be a physical connection in hardware. The communication port 720 may be configured to connect with a network 750, external media, the display 710, or any other components in system 700, or combinations thereof. The connection with the network 750 may be a physical connection, such as a wired Ethernet connection or may be established wirelessly as discussed below. Likewise, the additional connections with other components of the system 700 may be physical connections or may be established wirelessly. The network 750 may alternatively be directly connected to the bus 708.

While the computer-readable medium 722 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 722 may be non-transitory, and may be tangible.

The computer-readable medium 722 can include a solid-state memory such as a memory card or other package that houses one or more non-volatile read-only memories. The computer-readable medium 722 can be a random-access memory or other volatile re-writable memory. Additionally or alternatively, the computer-readable medium 722 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 700 may be connected to one or more networks 750. The network 750 may define one or more networks including wired or wireless networks. The wireless network may be a cellular telephone network, an 802.11, 802.16, 802.20, or WiMax network. Further, such networks may include a public network, such as the Internet, a private network, such as an intranet, or combinations thereof, and may utilize a variety of networking protocols now available or later developed including, but not limited to TCP/IP based networking protocols. The network 750 may include wide area networks (WAN), such as the Internet, local area networks (LAN), campus area networks, metropolitan area networks, a direct connection such as through a Universal Serial Bus (USB) port, or any other networks that may allow for data communication. The network 750 may be configured to couple one computing device to another computing device to enable communication of data between the devices. The network 750 may generally be enabled to employ any form of machine-readable media for communicating information from one device to another. The network 750 may include communication methods by which information may travel between computing devices. The network 750 may be divided into sub-networks. The sub-networks may allow access to all of the other components connected thereto or the sub-networks may restrict access between the components. The network 750 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method comprising:
receiving, by one or more first processors, pet data associated with an entity from one or more data sources;
receiving, by the one or more first processors, genetic test results data from a genetic test results database;
analyzing, by the one or more first processors, the pet data received from the one or more data sources with the genetic test results data received from the genetic test results database using an interpretation logic data structure;
determining, by the one or more first processors, a result value associated with the pet data and the genetic test results data based on the analysis;
determining, by the one or more first processors, one or more result statements associated with the result value; and
generating, by the one or more first processors, one or more health reports based on the one or more result statements.

2. The method of claim 1, wherein the interpretation logic data structure:
comprises a plurality of result values and a combination of result statements associated with each result value; and
defines a genetic test results range, a periodontal disease risk range, and a halitosis status for each of the plurality of result values.

3. The method of claim 2, wherein analyzing the pet data received from the one or more data sources with the genetic test results data received from the genetic test results database using the interpretation logic data structure comprises:
comparing the pet data and the genetic test results with the genetic test results range, the periodontal disease risk range, and the halitosis status defined for each of the plurality of result values.

4. The method of claim 2, wherein each of the combination of result statements comprises at least one of: a diagnosis or an oral care recommendation.

5. The method of claim 1, wherein each of the one or more health reports comprises a corresponding result statement of the one or more result statements.

6. The method of claim 1, further comprising:
transmitting, by the one or more first processors, the one or more health reports to one or more user devices.

7. The method of claim 1, wherein the one or more health reports comprise a pet owner report and/or a veterinarian report.

8. The method of claim 1, wherein the pet data comprises halitosis data and periodontal disease risk data.

9. The method of claim 8, wherein the periodontal disease risk data is obtained from a periodontal disease risk determination component.

10. The method of claim 9, wherein the periodontal disease risk determination component is configured to:
receive, by one or more second processors, one or more periodontal disease attributes from a database or one or more user devices;
analyze, by the one or more second processors, the one or more periodontal disease attributes;
determine, by the one or more second processors, a periodontal disease risk level based on the analysis; and
generate, by the one or more second processors, the periodontal disease risk data based on the determined periodontal disease risk level.

11. The method of claim 10, wherein the one or more periodontal disease attributes comprise at least one of: breed, size category, weight, body condition, age, shape of head, predicted size category, predicted adult weight, amount of plaque, amount of tartar, or bacterial composition of plaque.

12. The method of claim 1, wherein the one or more first processors are implemented on a cloud platform.

13. A system comprising:
a genetic test results database;
an interpretation logic data structure;
a periodontal disease risk determination component executed by one or more first processors and configured to:
receive one or more periodontal disease attributes from a database or one or more user devices;
analyze the one or more periodontal disease attributes;
determine a periodontal disease risk level based on the analysis; and
generate periodontal disease risk data based on the determined periodontal disease risk level; and
an analytics module executed by one or more second processors and configured to:
receive pet data associated with an entity from one or more data sources, the pet data including at least the periodontal disease risk data from the periodontal disease risk component;
receive genetic test results data from the genetic test results database;
analyze the pet data received from the one or more data sources with the genetic test results received from the genetic test results database using the interpretation logic data structure; and
determine a result value associated with the pet data and the genetic test results data based on the analysis.

14. The system of claim 13, wherein the result value is associated with one or more result statements.

15. The system of claim 14, wherein the analytics module is further configured to:
determine the one or more result statements associated with the result value; and
generate one or more health reports based on the one or more result statements.

16. The system of claim 13, wherein the pet data further includes halitosis data.

17. The system of claim 13, wherein the one or more periodontal disease attributes comprise at least one of: breed, size category, weight, body condition, age, shape of head, predicted size category, predicted adult weight, amount of plaque, amount of tartar, bacterial composition of plaque, or pet oral care routine.

18. The system of claim 13, wherein the pet data further includes signs of periodontal disease, the signs of periodontal disease including the presence of plaque, bleeding gums, tartar, change in behavior, sensitive stomach, halitosis, or a combination thereof.

19. The system of claim 13, wherein the analytics module is implemented on a cloud platform.

20. A non-transitory computer readable medium storing instructions that, when executed by one or more processors, cause the one or more processors to:
receive pet data associated with an entity from one or more data sources;
receive genetic test results data from a genetic test results database;
analyze the pet data received from the one or more data sources with the genetic test results data received from the genetic test results database using an interpretation logic data structure;
determine a result value associated with the pet data and the genetic test results data based on the analysis;
determine one or more result statements associated with the result value; and
generate one or more health reports based on the one or more result statements.
